(19) United States
(12) Patent Application Publication
(10) Pub. No.: US 2022/0336811 A1
(43) Pub. Date: Oct. 20, 2022

(54) CATHODE ACTIVE MATERIAL FOR SECONDARY BATTERY COMPRISING CHARGE TRANSFER COMPLEX AND METHOD FOR MANUFACTURING SAME

(71) Applicant: SEOUL NATIONAL UNIVERSITY R&DB FOUNDATION, Seoul (KR)

(72) Inventors: Ki Suk KANG, Seoul (KR); Se Chan LEE, Chungcheongbuk-do (KR); Ji Hyun HONG, Seoul (KR)

(21) Appl. No.: 17/634,314

(22) PCT Filed: Aug. 13, 2020
(86) PCT No.: PCT/KR2020/095100

§ 371 Date: Feb. 10, 2022

(30) Foreign Application Priority Data
Aug. 14, 2019 (KR) 10-2019-0099648

(51) Int. Cl. H01M 4/60; H01M 10/0525; C07C 255/31; C07D 241/46

(52) U.S. Cl. CPC H01M 4/60; H01M 10/0525; C07C 255/31; C07D 241/46; H01M 2004/028

(57) ABSTRACT

Provided are a cathode active material for secondary batteries, the cathode active material comprising a charge-transfer complex in which an electron donor and an electron acceptor are bonded to each other, wherein the electron donor and the electron acceptor are bonded to each other by intermolecular interaction, and a method of producing the cathode active material.

### TECHNICAL FIELD

The present invention relates to a cathode active material for secondary batteries, the cathode active material including an organic charge-transfer complex (OCTC), and a method of producing the cathode active material.

### BACKGROUND ART

Various types of organic electrode materials are being developed in response to the demand for next-generation lithium-ion batteries. The organic electrode materials use an organic material instead of a metal salt such as lithium cobalt oxide, and thus a reduction in production cost and a reduction in weight of secondary batteries are expected. In addition, because molecules may be designed to enable multi-electron reaction of an organic cathode active material, an increase in battery capacity may also be expected.

However, an intrinsically low electrical conductivity of the organic material and elution to an organic electrolyte solvent deteriorate a rate capability and a cycle performance, respectively. To solve these problems, a method of polymerizing organic redox-active monomers or a method of synthesizing a composite by using a conductive scaffold has been proposed. Polymerization may alleviate the elution problem by fixing the redox-active components to an insoluble polymer, but causes a lower electrical conductivity and an increased weight, and deterioration of power capacity and gravimetric energy density due to an increase of redox-inactive parts. When the composite is synthesized using a carbon-based conductive scaffold, although conductivity is increased and power and capacity retention are improved, an electrode weight is increased to reduce a specific energy density, and long-term charge/discharge cyclability is not stable. As described above, a material group capable of simultaneously solving a low rate capability and a low cycle performance has not been developed.

### DETAILED DESCRIPTION OF THE INVENTION

### Technical Problem

The present invention provides an electrode material capable of achieving enhanced electrochemical properties by increasing an intrinsically low electrical conductivity of an organic material and reducing a solubility based on strong intermolecular bonds. However, the above problem to be solved is merely an example and the scope of the present invention is not limited thereto.

### Technical Solution

According to an aspect of the present invention, there is provided a cathode active material for secondary batteries. The cathode active material for secondary batteries may include an organic charge-transfer complex (OCTC) in which an electron donor and an electron acceptor are bonded to each other, and the electron donor and the electron acceptor may be bonded to each other by intermolecular interaction.

In the cathode active material for secondary batteries, the electron donor may be phenazine (PNZ) represented by Chemical Formula 1.

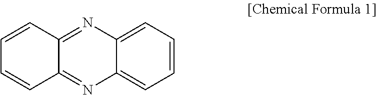 [SMILES: c1ccc2nc3ccccc3nc2c1]

In the cathode active material for secondary batteries, the electron acceptor may be 7,7,8,8-tetracyanoquinodimethane (TCNQ) represented by Chemical Formula 2.

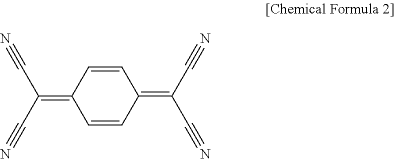 [SMILES: N#CC(C#N)=c1ccc(=C(C#N)C#N)cc1]

In the cathode active material for secondary batteries, the electron donor may be phenazine (PNZ), and the electron acceptor may be 7,7,8,8-tetracyanoquinodimethane (TCNQ).

In the cathode active material for secondary batteries, the OCTC may include two or more stacked layers, and π-π interaction may be present between aromatic rings of the electron donor included in a layer and the electron acceptor included in an adjacent layer.

According to an aspect of the present invention, there is provided a secondary battery. The secondary battery may include a cathode including the above-described cathode active material for secondary batteries, an anode, and an electrolyte layer.

According to an aspect of the present invention, there is provided a method of producing a cathode active material for secondary batteries. The method of producing the cathode active material for secondary batteries may include mixing an electron donor and an electron acceptor, and forming an organic charge-transfer complex (OCTC) in which the electron donor and the electron acceptor are bonded to each other by intermolecular interaction.

In the method of producing the cathode active material for secondary batteries, the electron donor may be phenazine (PNZ), and the electron acceptor may be 7,7,8,8-tetracyanoquinodimethane (TCNQ).

In the method of producing the cathode active material for secondary batteries, the electron donor and the electron acceptor may be mixed in a molar ratio of 1:0.9 to 1:1.1.

### Advantageous Effects

According to the afore-described embodiments of the present invention, a rate capability and a cycle performance may be improved based on a high electrical conductivity and a low solubility. However, the scope of the present invention is not limited to the above-described effect.

### DESCRIPTION OF THE DRAWINGS

FIG. 1 is a conceptual view of a cathode active material for secondary batteries, according to an embodiment of the present invention.

FIG. 2 shows the operating principle of a cathode active material for secondary batteries, according to an embodiment of the present invention.

FIG. 3 comparatively shows electrical conductivities of a cathode active material for secondary batteries, according to an embodiment of the present invention, and other organic/inorganic redox-active materials.

FIG. 4 shows the structure and a scanning electron microscope (SEM) image of a cathode active material for secondary batteries, the cathode active material including phenazine (PNZ)-7,7,8,8-tetracyanoquinodimethane (TCNQ), according to an embodiment of the present invention.

FIG. 5 shows X-ray diffraction (XRD) patterns of a cathode active material for secondary batteries, the cathode active material including PNZ-TCNQ, according to an embodiment of the present invention, and monomers thereof.

FIG. 6 shows solubilities of a cathode active material for secondary batteries, the cathode active material including PNZ-TCNQ, according to an embodiment of the present invention, and monomers thereof.

FIG. 7 shows charge/discharge profiles of lithium half-cells made of a cathode active material for secondary batteries, the cathode active material including PNZ-TCNQ, according to an embodiment of the present invention, and monomers thereof.

FIG. 8 shows a rate capability of a cathode active material for secondary batteries, the cathode active material including PNZ-TCNQ, according to an embodiment of the present invention.

FIG. 9 shows a voltage-capacity profile of a cathode active material for secondary batteries, the cathode active material including PNZ-TCNQ, according to an embodiment of the present invention, based on a concentration.

FIG. 10 shows capacity retention of a cathode active material for secondary batteries, the cathode active material including PNZ-TCNQ, according to an embodiment of the present invention.

FIGS. 11A, 11B and 11C show a result of quantitatively analyzing a solubility of a cathode active material for secondary batteries, the cathode active material including PNZ-TCNQ, according to an embodiment of the present invention.

FIG. 12 shows a discharge profile of a cathode active material for secondary batteries, the cathode active material including PNZ-TCNQ, according to an embodiment of the present invention.

FIG. 13 comparatively shows cyclabilities of a charge-transfer complex of a cathode active material for secondary batteries, the cathode active material including PNZ-TCNQ, according to an embodiment of the present invention, and monomers thereof.

### MODE OF THE INVENTION

Hereinafter, the present invention will be described in detail by explaining embodiments of the invention with reference to the attached drawings. The invention may, however, be embodied in many different forms and should not be construed as being limited to the embodiments set forth herein; rather, these embodiments are provided so that this disclosure will be thorough and complete, and will fully convey the concept of the invention to one of ordinary skill in the art. In the drawings, the thicknesses or sizes of layers are exaggerated for clarity and convenience of explanation.

A cathode active material for secondary batteries, according to the present invention, will now be described in detail.

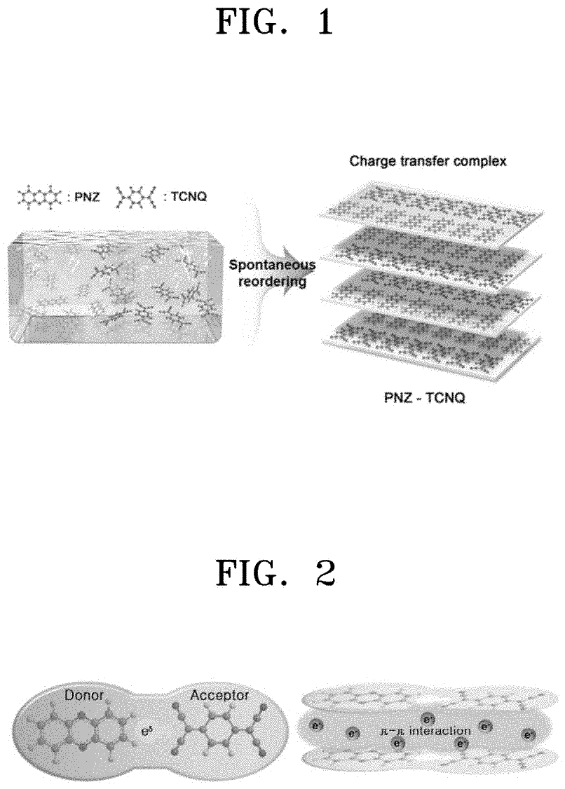

FIG. 1 is a conceptual view of a cathode active material for secondary batteries, according to an embodiment of the present invention.

In FIG. 1, the cathode active material for secondary batteries, according to an embodiment of the present invention, includes an organic charge-transfer complex (OCTC) to solve a low electrical conductivity and a solubility reduction without losing the intrinsic redox ability of an organic compound. The OCTC is a combination of two or more organic molecules with different electron accepting abilities, and some of electronic charges may be delivered between the molecules.

The OCTC according to an embodiment of the present invention may be bound by interaction based on non-covalent bonds between organic molecules of an electron donor and an electron acceptor. In an embodiment, the electron donor and the electron acceptor may form a molecular layer through strong hydrogen bonds and thus a high structural stability may be provided.

In the OCTC, the electron donor may be phenazine (PNZ) represented by Chemical Formula 1.

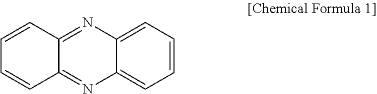 [SMILES: c1ccc2nc3ccccc3nc2c1]

In the OCTC, the electron acceptor may be 7,7,8,8-tetracyanoquinodimethane (TCNQ) represented by Chemical Formula 2.

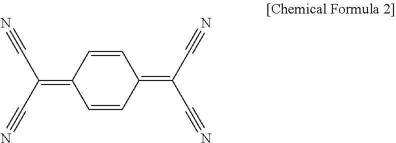 [SMILES: N#CC(C#N)=c1ccc(=C(C#N)C#N)cc1]

The cathode active material for secondary batteries, according to an embodiment of the present invention, may include the OCTC in which PNZ and TCNQ are bonded to each other. PNZ and TCNQ may be bonded to each other by intermolecular hydrogen bonds and thus form a molecular layer.

The OCTC may include two or more stacked molecular layers. π-π interaction may be present between aromatic rings of the electron donor included in a molecular layer and the electron acceptor included in an adjacent molecular layer. The layered structure of the OCTC may be well-ordered by π-π interaction between the molecular layers.

FIG. 2 shows the operating principle of a cathode active material for secondary batteries, according to an embodiment of the present invention.

In FIG. 2, high-density electron cloud formed between molecular layers by π-π interaction therebetween may serve as a charge-transfer path through which electrons may move freely. An OCTC in which an electron donor and an electron acceptor are bonded to each other may greatly increase electrical conductivity compared to each of the molecules constituting the complex.

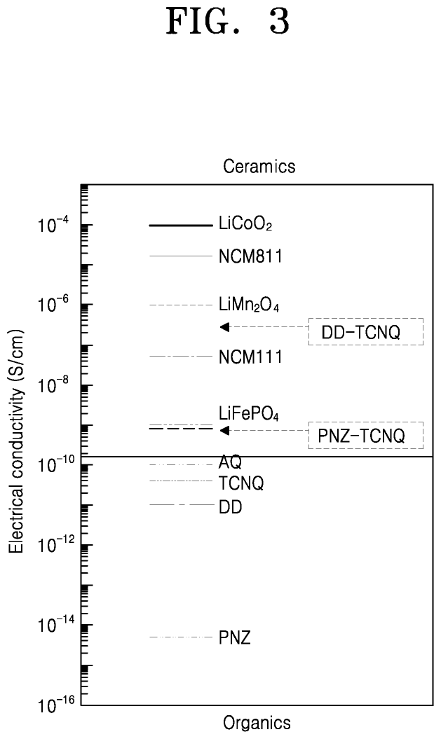

FIG. 3 comparatively shows electrical conductivities of a cathode active material for secondary batteries, according to an embodiment of the present invention, and other organic/inorganic redox-active materials.

In FIG. 3, an electrical conductivity of an OCTC in which PNZ and TCNQ are bonded to each other is increased compared to the electrical conductivity of PNZ or TCNQ.

PNZ and TCNQ may be organic redox-active compounds, and PNZ may provide redox activity at 1.5/1.2V (vs. Li/Li⁺) whereas TCNQ may provide redox activity at 3.2/2.6V (vs. Li/Li⁺).

In an embodiment of the present invention, an electron donor may be PNZ, and an electron acceptor may be TCNQ. A planar structure of PNZ may contribute to formation of a layered crystalline structure in the OCTC. The OCTC based on PNZ-TCNQ may be formed by intermolecular interaction through a simple mixing process at room temperature. Strong intermolecular bonds between PNZ and TCNQ may facilitate formation of the PNZ-TCNQ OCTC in a high yield at room temperature, and contribute to stability of the crystal structure of the OCTC.

In a method of producing the cathode active material for secondary batteries, the electron donor and the electron acceptor may be mixed in a molar ratio of 1:0.9 to 1.1, and more specifically, in a molar ratio of 1:1. When the above-mentioned range is not satisfied, the OCTC may not be formed and thus the electrical conductivity may be lowered.

Test examples for verifying properties of the cathode active material for secondary batteries, according to the present invention, will now be described. However, the following test examples are merely for better understanding of the present invention, and embodiments of the present invention are not limited thereto.

### Embodiment 1

To synthesize an OCTC, organic precursor materials such as PNZ and TCNQ were prepared, and then these two types of organic materials were mixed in an equimolar ratio, and dissolved and stirred in an acetone solvent at room temperature for 3 hours. The obtained solution was filtered in a vacuum by using an inorganic filter with a pore size of 10 μm, and then a precipitate on the filter was stored in a 30° C. vacuum oven overnight to obtain a final product.

### COMPARATIVE EXAMPLE 1

99%-purity PNZ powder was purchased from Alfa Aesar.

### COMPARATIVE EXAMPLE 2

98%-purity TCNQ powder was purchased from Sigma-Aldrich.

### Embodiment 2

Cathode mixtures were produced by mixing 40 wt % of the cathode active materials according to Embodiment 1 and Comparative Examples 1 and 2, 40 wt % of a conductor such as Super P, and 20 wt % of a binder such as polytetrafluoroethylene (PTFE). The synthesized cathode mixtures were rolled with a stainless steel (SUS) rod and cut to a size of 1.5 cm×1.5 cm to produce cathodes for secondary batteries.

### Embodiment 3

A porous polyethylene separator was placed between a lithium-based anode and the cathodes for secondary batteries, which were produced in Embodiment 2, and a lithium electrolyte was injected to produce coin-type lithium half-cells.

### TEST EXAMPLE 1

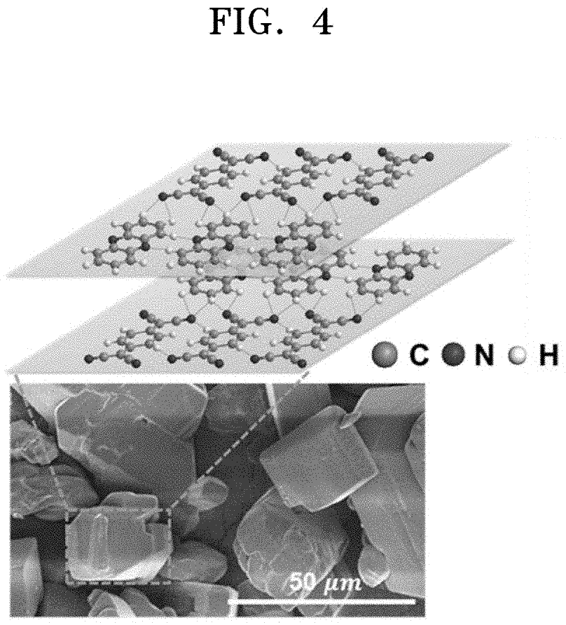

A scanning electron microscope (SEM) image of the OCTC formed according to Embodiment 1 is shown in FIG. 4. It implies that a new crystalline phase is formed after PNZ and TCNQ are mixed and dried. In the molecular structure model of FIG. 4, hydrogen bonds between nitrogen and hydrogen atoms of PNZ and TCNQ are indicated by dashed lines. PNZ and TCNQ may be bonded to each other by intermolecular hydrogen bonds to form a single molecular layer. In addition, the OCTC may have a structure in which two or more molecular layers are stacked on one another, and a π-π conjugation region may be formed due to intermolecular attractive force between layers to maintain a well-ordered stacked structure.

### TEST EXAMPLE 2

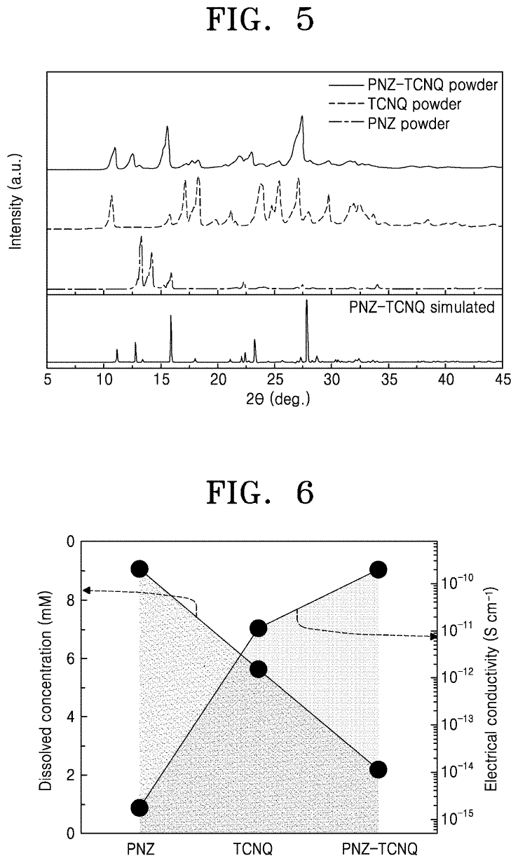

X-ray diffraction (XRD) patterns of the cathode active materials according to Embodiment 1 and Comparative Examples 1 and 2 are shown in FIG. 5. It is shown that the synthesized material of Embodiment 1 has a new phase different from the phases of the constituent molecules of Comparative Examples 1 and 2, and this result is consistent with a simulated XRD pattern based on an ordered PNZ-TCNQ layer structure.

### TEST EXAMPLE 22

16-pi disc pellets were produced using the cathode active material powders according to Embodiment 1 and Comparative Examples 1 and 2, and 4-probe measurement was performed. A result of measuring electrical conductivities thereof is shown in a line graph of FIG. 6. The electrical conductivity of the cathode active material of Embodiment 1 is 8×10-10 S cm⁻¹, which is about 105 times and about 35 or 36 times higher than those of Comparative Examples 1 and 2, and it shows that the electrical conductivity is rapidly increased through a simple structural change between molecules. It is regarded that this is because a fast charge-transfer path is formed due to π-π interaction between layers.

### TEST EXAMPLE 3

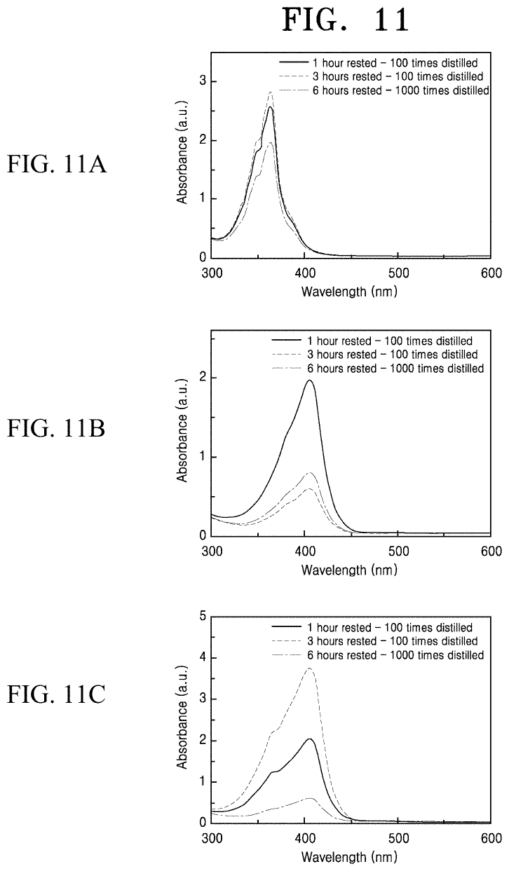

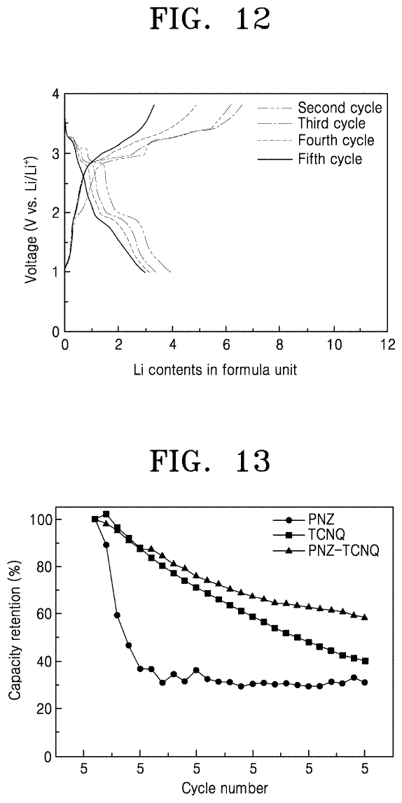

The cathodes produced in Embodiment 2 were put in 4 mL of a tetraethyleneglycol dimethylether (TEGDME) solvent, and stored in a 60° C. oven for 3 hours, and then solubilities of the cathode active materials for a solvent in an electrolyte were measured through ultraviolet-visible (UV-Vis) spectroscopy by using the used solvent. The solubilities calculated based on the Beer-Lambert law are shown in FIGS. 6 and 11. The cathode active material according to Embodiment 1 exhibits a solubility of about 2.267 mM whereas the cathode active materials according to Comparative Examples 1 and 2 exhibit solubilities of 9.087 mM and 5.625 mM, respectively, and it shows that the OCTC effectively reduces the solubility. This is because of strong intermolecular interaction between the electron donor and the electron acceptor in the OCTC.

### TEST EXAMPLE 4

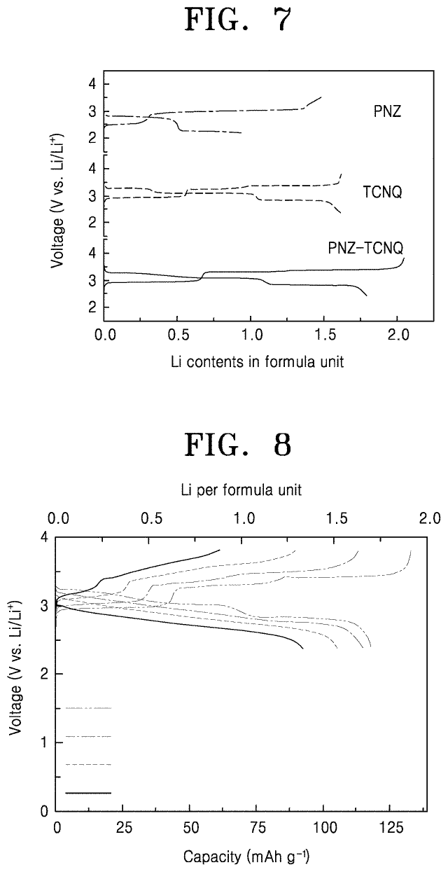

Charge/discharge results of the coin-type lithium half-cells according to Embodiment 3 in a second cycle are shown in FIG. 7. The cathode active material of Embodiment 1 exhibits a high theoretical capacity utilization rate of about 90% compared to the cathode active materials according to Comparative Examples 1 and 2 corresponding to the constituent molecules (50% and 75%, respectively), and it shows that a rate capability and an oxidation/reduction rate in the active material structure are increased.

### TEST EXAMPLE 5

To verify the rate capability of Embodiment 1, charge/discharge results at various current rates are shown in FIG. 8. Embodiment 1 exhibits a high discharge capacity corresponding to 60% of a theoretical capacity even at a high current rate of 500 mA g⁻¹, and 73% of a capacity implemented at 50 mA g⁻¹, and it shows that electrochemical properties are enhanced based on the increased electrical conductivity. FIG. 8 shows that the rate capability of the OCTC is remarkably increased. The electrochemical profile of the OCTC electrode at various current rates shows that retention of a specific capacity is relatively stable even when a current density is increased from 20 mA g⁻¹ to 500 mA g⁻¹.

### TEST EXAMPLE 6

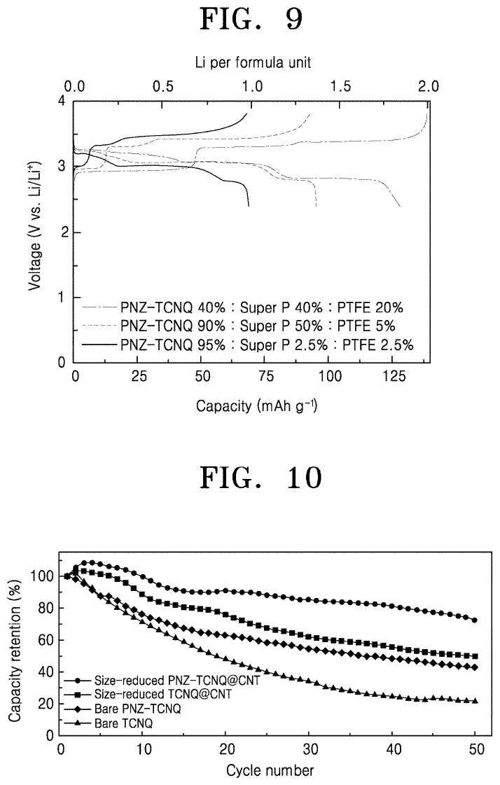

Organic electrodes generally have a low content of an active material in the electrode (e.g., 20% to 60%) to compensate for deterioration of electrical conductivity, and thus a charge and discharge profile of an OCTC electrode including conductive carbon is shown in FIG. 9. When the content of carbon is reduced, the capacity of the OCTC electrode is gradually reduced.

### TEST EXAMPLE 7

A line graph for comparing cycle performances of the cathode active materials according to Embodiment 1 and Comparative Examples 1 and 2 is shown in FIG. 13. A solubility lower than those of the cathode active materials according to Comparative Examples 1 and 2 affects the cycle performance and thus Embodiment 1 exhibits an improved cycle performance. A capacity retention of the OCTC electrode after 50 cycles is 43%, which is remarkably higher than that of the TCNQ electrode (21%). Therefore, it is clearly proved that formation of the OCTC increases the cycle retention compared to each of the organic molecules constituting the OCTC. Overall cycle performance may be improved by solving a large particle size observed in the image of FIG. 4 and a small amount of elution observed in FIG. 6.

While the present invention has been particularly shown and described with reference to embodiments thereof, it will be understood by one of ordinary skill in the art that various changes in form and details may be made therein without departing from the scope of the present invention as defined by the following claims.

1. A cathode active material for secondary batteries, the cathode active material comprising an organic charge-transfer complex (OCTC) in which an electron donor and an electron acceptor are bonded to each other,
wherein the electron donor and the electron acceptor are bonded to each other by intermolecular interaction.

2. The cathode active material of claim 1, wherein the electron donor is phenazine (PNZ) represented by Chemical Formula 1.

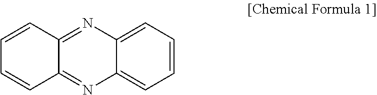 [SMILES: c1ccc2nc3ccccc3nc2c1]

3. The cathode active material of claim 1, wherein the electron acceptor is 7,7,8,8-tetracyanoquinodimethane (TCNQ) represented by Chemical Formula 2.

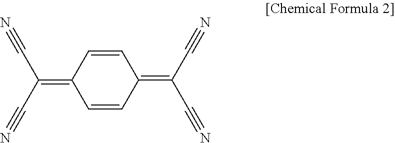 [SMILES: N#CC(C#N)=c1ccc(=C(C#N)C#N)cc1]

4. The cathode active material of claim 1, wherein the OCTC comprises two or more stacked layers, and
wherein π-π interaction is present between the electron donor comprised in a layer and the electron acceptor comprised in an adjacent layer.

5. A secondary battery comprising:
a cathode comprising the cathode active material of claim 1;
an anode; and
an electrolyte layer.

6. A method of producing a cathode active material for secondary batteries, the method comprising:
mixing an electron donor and an electron acceptor; and
forming an organic charge-transfer complex (OCTC) in which the electron donor and the electron acceptor are bonded to each other by intermolecular interaction.

7. The method of claim 6, wherein the electron donor is phenazine (PNZ), and the electron acceptor is 7,7,8,8-tetracyanoquinodimethane (TCNQ).

8. The method of claim 6, wherein the electron donor and the electron acceptor are mixed in a molar ratio of 1:0.9 to 1:1.1.

9. A secondary battery comprising:
a cathode comprising the cathode active material of claim 2;
an anode; and
an electrolyte layer.

10. A secondary battery comprising:
a cathode comprising the cathode active material of claim 3;
an anode; and
an electrolyte layer.

11. A secondary battery comprising:
a cathode comprising the cathode active material of claim 4;
an anode; and
an electrolyte layer.
